SEQUENCE LISTING (2 sequences)

SEQ ID NO: 1 - DNA, 20 bp, Artificial sequence
  Synthetic oligonucleotide ODN.
ttcatgtctctgctggtcgc

SEQ ID NO: 2 - DNA, 20 bp, Artificial sequence
  Synthetic oligonuceotide ODN
gtacgtctgttcctgttccg

(19) United States
(12) Patent Application Publication
(10) Pub. No.: US 2008/0287381 A1
(43) Pub. Date: Nov. 20, 2008

(54) Injectable Agent for the Targeted Treatment of Retinal Ganglion Cells

(76) Inventors: Sebastian Thaler, Ammerbuch (DE); Frank Schuttauf, Tubingen (DE)

Correspondence Address: KLARQUIST SPARKMAN, LLP, 121 SW SALMON STREET, SUITE 1600, PORTLAND, OR, 97204, US

(21) Appl. No.: 11/815,008

(22) PCT Filed: Jan. 25, 2006
(86) PCT No.: PCT/EP2006/000622

§ 371 Date: Mar. 11, 2008

(30) Foreign Application Priority Data
Jan. 30, 2005 (DE) 10 2005 005 528.1

(51) Int. Cl. A61K 31/7088; A61P 27/02

(52) U.S. Cl. 514/44

(57) ABSTRACT

The present invention relates to the targeted treatment of retinal ganglion cells (RGC).

The present invention relates to the targeted treatment of retinal ganglion cells (RGC).

The targeted treatment of disorders of the retina such as, for example, glaucoma, diabetic retinopathy, represents one of the greatest challenges to medicine and pharmaceutical research in the area of opthalmology, i.e. of modern medicine relating to the eye. A particular aim in this connection is to provide a substance or a composition which enters the retinal ganglion cells, i.e. the nerve cells of the vertebrate eye whose axons leave the eye via the optic nerve. It is important in this connection that such a substance or such a composition enters the retinal ganglion cells exclusively and in a targeted manner and is able to display its activity there where appropriate, but not in other regions of the eye or of the retina. Only in this way a selective treatment of the disorder of retinal ganglion cells is possible without other tissues or cells of the organism or eye having their physiological function impaired or damaged. Side effects of a corresponding treatment of a patient are thus reduced.

Shen et al. (2002), “Preclinical evaluation of a phosphorothioate oligonucleotide in the retina of rhesus monkey”, Lab. Invest. 82, pages 167 to 182, describe an oligonucleotide which can be injected into the corpus vitreum, i.e. the vitreous body of the eye, or into subretinal regions, and can thus be introduced into the retina.

Similar data on experiments in which substances directly injectable into eyes are described had previously been presented by Shen, W. Y. and Rakoczy, W. E. (2001), “Uptake dynamics and retinal tolerance of phosphorothioate oligonucleotide and its direct delivery into the site of choroidal neovascularization through subretinal administration in the rat”, Antisense Nucleic Acid Drug Dev. 11, pages 257 to 264, and Vorwerk et al. (2000), “Depression of retinal glutamate transporter function leads to elevated intravitreal glutamate levels and ganglion cell death”, Invest. Opthalmol. Vis. Sci. 41, pages 3615 to 3621.

Injections into the eye, specifically into the corpus vitreum, which are also referred to as intravitreal injections, are disadvantageous, however. Thus, such an injection is associated with the risk of extensive damage to the lens of the eye. This administration may be associated with bleeding at the puncture site or with infections affecting the whole eye. There is also the risk that the lens of the eye will be damaged, possibly resulting in the modulation of neuroprotective metabolic pathways, thus greatly impairing investigations and therapies in this region, or making them entirely impossible.

A further entirely decisive disadvantage of substances which can be injected intravitreally, such as oligonucleotides, is that, following injection thereof, they are transported nonspecifically into more or less all retinal cell layers and other regions of the organism, and therefore selective transport of the substances to the retinal ganglion cells is impossible. Substances which can be injected intravitreally are therefore unsuitable for targeted treatment of disorders of the retinal ganglion cells.

Husak et al. (2000), “Pseudorabies virus membrane proteins gI and gE facilitate anterograde spread of infection in projection-specific neurons in the rat”, J. Virol. 74, pages 10975 to 10983, describe virus particles and viral vectors which can be injected into regions of the midbrain, i.e. into the superior colliculus, and by a subsequent retrograde transport in the tissue of the brain reach the retina and the retinal ganglion cells.

Comparable virus particles or viral constructs are described by Kaspar et al. (2002), “Targeted retrograde gene delivery for neuronal protection”, Mol. Ther. 5, pages 50 to 56, and by Peltekian et al. (2002), “Neurotropism and retrograde axonal transport of a canine adenoviral vector: a tool for targeting key structures undergoing neurodegenerative processes”, Mol. Ther. 5, pages 25 to 32.

However, viral vectors are extremely problematic for therapeutic or diagnostic use because, after introduction into the organism, they cause a large number of virus-specific side effects which may extend to the induction of apoptosis. Moreover, the production of such viral constructs is associated with high costs and relatively great complexity, so that there is no question of use on a large scale. Viral vectors and virus particles are therefore likewise unsuitable for targeted treatment of disorders of the retina.

It is therefore an object of the present invention to provide an RGC-specific composition which can be injected into the brain of a living being and with which the prior art disadvantages are avoided. It is particularly intended to provide such a composition which has therapeutic and diagnostic potential and can be transported in targeted fashion into retinal ganglion cells. This composition should be simple to produce and, after injection into the brain of a living being, accumulate mainly in the retinal ganglion cells and not, or only to a slight and tolerable extent, in other retinal cell layers or in other cells of the organism.

This object is achieved through the use of a nucleic acid molecule for producing an injectable composition which is transported in targeted fashion into retinal ganglion cells.

Specifically, the inventors have surprisingly found, with the assistance of a rat model, that an isolated nucleic acid molecule, for example an oligonucleotide, is transported in targeted fashion into retinal ganglion cells after injection into the brain of a mammal. It was particularly surprising in this connection that such a nucleic acid molecule requires no viral components whatsoever in order to reach the RGC by retrograde transport. It was astonishing to observe that such a nucleic acid molecule is to be found exclusively in the RGC, whereas, following injection of nucleic acid molecules into the brain, none of the latter were detectable in other retinal cell layers or other cells of the organism.

Such a result was not to be expected. On the contrary, it has to date been assumed on the basis of the data presented by Husak et al. and Kasper et al (loc. cit.) that only specific viral constructs or whole virus particles are transported into retinal ganglion cells after injection into the brain. However, a nucleic acid molecule is advantageous in many respects by comparison with a virus particle or viral construct. Thus, such as molecule is considerably simpler to prepare, is distinguished by increased stability and is particularly suitable both as actual active substance or else as carrier molecule for an active substance coupled thereto. It is particularly advantageous on use of a nucleic acid molecule that the latter does not induce any virus-specific side effects in the organism and therefore is particularly suitable as therapeutic agent.

A nucleic acid molecule means a molecule which includes both oligonucleotides and polynucleotides with any desired nucleotide sequences. A nucleic acid molecule therefore means according to the invention linear or else branched-chain di-, tri- etc. -nucleotides, connected by 3′,5′-phosphodiester linkages, up to an unlimited chain length. Such nucleic acid molecules can be prepared easily by targeted nucleotide synthesis or by incomplete enzymatic or chemical cleavage of nucleic acids. Such methods are generally known in the state of the art and familiar to the skilled worker; cf. Sambrook, J. and Russell, D. W. (2001), “Molecular Cloning—A Laboratory Manual”, Cold Spring Harbor Laboratory Press, New York. The content of this publication is incorporated in the present application by reference.

In particular, a nucleic acid molecule does not mean according to the invention a virus particle or else viral construct such as a viral vector.

The inventors have realized in this connection that the nucleotide sequence of the nucleic acid molecule is not crucial for the invention, although certain sequences may, where appropriate, have advantages in relation to the desired therapeutic or diagnostic use of the composition. The inventors have on the contrary recognized and technically exploited a general principle by means of which, irrespective of the nucleotide sequence of the nucleic acid molecule employed, targeted therapy and diagnosis of disorders of the retinal ganglion cells are possible.

A targeted transport into retinal ganglion cells means according to the invention that other cell types, especially other cell layers of the retina, remain substantially unaffected, i.e. injection of the nucleic acid molecule into the brain leads to substantially exclusive transport of the latter into retinal ganglion cells. Allowance is made in this connection for small amounts of the nucleic acid molecule being transported where appropriated into non-RGC or other cell types, but the latter being extremely small by comparison with the amount of nucleic acid molecules transported in targeted manner into the RGC, and therefore tolerable in relation to the advantages.

A composition means according to the invention any composition such as a pharmaceutical composition or a medicament or a diagnostic composition and a material to be used in research which either consists exclusively of nucleic acid molecule or, where appropriate, additionally comprises a buffer substance, a pharmaceutically or diagnostically acceptable carrier or else at least one further active ingredient, diagnostic marker or other excipient.

A composition of the invention may also be used outside a living being, for example a scientific tool which can be introduced or injected into isolated cell or tissue cultures which include retinal ganglion cells.

It is preferred in this connection according to the invention for the composition to be designed for injection into the superior colliculus.

The superior colliculus is an organ which forms part of the midbrain tectum. The nerve tracts leading to this organ carry inter alia visual signals and form therein representations of the visual field.

The inventors have surprisingly been able to show that a nucleic acid molecule-containing composition designed in this way, that the latter can be injected into the superior colliculus of a mammalian brain and, after the injection, is transported by retrograde transport, i.e. from the periphery of a nerve cell in the direction of the cell body, exclusively into retinal ganglion cells.

This measure provides a composition which can be injected into an anatomically readily accessible and locatable structure of the midbrain, and in particular no hazardous injection into the eye is necessary for a selective administration of the composition or nucleic acid molecule into the retinal ganglion cells.

In a preferred refinement, an active ingredient is coupled to the nucleic acid molecule.

This measure has the particular advantage that the nucleic acid molecule is used as carrier substance, i.e. as so-called carrier, and virtually any desired active ingredient can be transported, via an appropriate coupling to the nucleic acid molecule, to the retinal ganglion cells and can there display for example a therapeutically utilizable activity. It is also possible thereby to transport into the retinal ganglion cells active ingredients which to date are distributed completely nonselectively in the organism and were therefore useless for a targeted therapy. There are sufficient descriptions in the prior art, cf. Sambrook and Russell (loc. cit.), of how a nucleic acid molecule, for example an oligonucleotide consisting of 10 to 30 nucleotides, can be coupled to any desired substance. This complex or a corresponding composition of the invention can then be injected through a fine needle into the superior colliculus, for example by means of stereotactic injections.

In a variant of the invention, the nucleic acid molecule itself is designed as active ingredient.

This measure has the advantage that for example the antisense technology which is established in the state of the art can be used for targeted manipulation of retinal ganglion cells. Coupling of a further active ingredient is thus superfluous. On the contrary, it is possible with the aid of databases which contain information about the nucleotide sequences of, where appropriate, RGC-specific genes or mRNA molecules of interest, to construct nucleic acid molecules which are able to interact with corresponding genetic information in the retinal ganglion cells and thus for example inhibit gene expression in these cells. It is advantageous in this connection that, as the inventors have been able to show, following injection of a nucleic acid molecule into the brain it is to be found in the retinal ganglion cells both in the cytoplasm, where it is able to interact for example with mRNA molecules, and in the nucleus, where it is able to interact for example with the genomic DNA.

It is preferred in this connection for the nucleic acid molecule to be in such a form that it modulates, preferably inhibits, gene expression in the retinal ganglion cells, and preferably has a nucleotide sequence which is substantially complementary to the sequence of an mRNA from the retinal ganglion cells.

This measure has the advantage that such a nucleic acid molecule hybridizes under stringent conditions with a corresponding complementary nucleic acid molecule, and thus inhibits the transcription of the genomic DNA and translation of the mRNA molecule, and ultimately leads to inhibition of the expression of the coding gene. It is thus possible in a simple and targeted manner to prevent overexpression of particular genes in retinal ganglion cells which are associated with the disorder of the eye or of the retina. A large number of such genes has now been sequenced and the sequences can be inspected in publicly accessible databases. It is possible on the basis of the nucleotide sequence of the genes to construct by routine measures a nucleic acid molecule which has an appropriate substantially complementary nucleotide sequence and is suitable for the use according to the invention.

The meaning of “substantially complementary” according to the invention is that the nucleic acid molecule is able to hybridize under stringent conditions with the mRNA from the retinal ganglion cells by forming hydrogen bonds. This is possible when the nucleic acid molecule has sufficiently long segments of a nucleotide sequence which is complementary to a nucleotide sequence of the mRNA. It is, however, unnecessary for the nucleic acid molecule to be able to hybridize over its entire length with the mRNA, i.e. have a nucleotide sequence which is complementary over the entire length. To this extent, substantially complementary also means in the sense of the invention that individual nucleotides within the nucleotide sequence of the nucleic acid molecule cannot form hydrogen bonds with the mRNA from the retinal ganglion cells, but the nucleic acid molecule as a whole is able to hybridize with the corresponding mRNA and thus to modulate gene expression. The hybridization properties of a nucleic acid molecule can easily be established by routine measures such as, for example construction of melting curves. Starting from the known nucleotide sequences of the genes whose expression is to be modulated, the skilled worker arrives with the aid of his expert knowledge and the carrying out of routine experiments, in a targeted manner at a nucleic acid molecule which is substantially complementary to the sequence of an mRNA from the retinal ganglion cells.

It is possible by this preferred embodiment to provide for example a nucleic acid molecule with which the disorders of the retina mentioned at the outset, namely glaucoma or diabetic retinopathy, can be treated. Thus, specifically, it is known that apoptotic processes take place in these disorders and in cases of ischemic damage and may ultimately lead to death of the ganglion cells. The apoptotic process in turn is mediated by the activity of various proteins such as, for example, c-fos, c-jun, p53, Bax, Apafl, caspase 9, caspase 3, caspase 6 PARP. A review of the proteins and genes involved in the apoptosis of ganglion cells is to be found in Nickells, R. W., 2004, The molecular biology of ganglion cell death: caveats and controversies, Brain Research Bulletin 62, pages 439 to 446. The sequences of these genes are known, so that it is easy for the skilled worker to prepare nucleic acid molecules having nucleotide sequences which are complementary to the nucleotide sequences of these genes or the mRNAs of the genes. It is possible with the aid of such nucleic acid molecules to inhibit the expression of the disease-mediating genes. Such nucleic acid molecules therefore represent a therapeutically valuable tool for treating such disorders of the retina.

It is preferred in this connection for the nucleic acid molecule to be in the form of siRNA.

Such siRNA molecules (small interfering RNA) represent double-stranded structures of ribonucleic acid. siRNA molecules are more suitable than simple antisense oligonucleotides for inhibiting gene expression because they are able to induce an autocatalytic post-transcriptional process which is referred to as RNA interference (RNAi) and leads to an extremely efficient shutting down of the expression of particular genes, called gene silencing. siRNA molecules introduced into a biological cell are recruited to form a so-called ribonuclease complex, called the RNA induced silencing complex (RISC). This complex is able, via the siRNA molecule, to bind to structures which are substantially complementary thereto, such as the mRNA of a gene transcribed in retinal ganglion cells, and to degrade them through the endonuclease activity of the RISC. The result thereof is inhibition of the expression of the corresponding gene which codes for the mRNA which is complementary to part of the siRNA molecule.

It is further preferred for the nucleic acid molecule to be configured in such a way that it binds to another nucleic acid molecule which codes for kynurenine aminotransferase II (KAT II) or parts thereof.

This measure has the advantage of providing a nucleic acid molecule which, following injection into the brain of a mammal, is transported especially well into retinal ganglion cells. The inventors have specifically constructed an oligonucleotide having the model nucleotide sequence TTCATGTCTCTGCTGGTCGC, where the 5′ end is located as usual on the left-hand side and the 3′ end is located on the right-hand side, which, after injection into the superior colliculus of a rat brain, is transported in a targeted manner into retinal ganglion cells. It was surprising in this connection that such a nucleic acid molecule is stable in the retinal ganglion cells over a lengthy time and there downregulates the expression of KAT II.

It is, of course, possible for a nucleic acid molecule of this or a comparable type to have according to the invention further nucleotide sequences at the 5′ or 3′ end without the targeted transport into the retinal ganglion cells being abolished thereby or the inhibition of the expression of KAT II being markedly reduced.

The inventors have proved by way of example with the aid of a model nucleic acid molecule having the nucleotide sequence described above that it is possible, starting from known nucleotide sequences which code for a gene of interest, to prepare an unlimited number of widely different nucleic acid molecules which can be injected into the superior colliculus and after retrograde transport, enter retinal ganglion cells in a targeted manner and there are able to inhibit or switch off the expression of the corresponding gene.

The inventors have therefore developed a generally valid technical concept which consists of the provision of a composition which can be used for therapy or diagnosis, which comprises nucleic acid molecules and with which the expression of any desired gene can be modulated or inhibited exclusively in retinal ganglion cells in a living being in a harmless manner without major surgical procedures. The invention is therefore not restricted to a particular nucleic acid molecule.

In a preferred variant, a detectable marker is coupled to the nucleic acid molecule.

This measure has the particular advantage of providing a composition with which the retrograde transport of substances to the retinal ganglion cells can be investigated for example as part of basic neurobiological research.

Suitable as marker is any substance which can be detected by known means of diagnosis or molecular biology, such as, for example, microscopy, fluorescence activated cell sorting (FACS), Western blotting, Northern blotting, autoradiography etc. Examples of such substances are fluorescent dyes such as Fluorogold, Cy3, or a peptide or protein such as peroxidase, biotin, streptavidin, avidin, alkaline phosphatase etc.

With this background, the present invention also relates to a nucleic acid molecule for treating an ocular disorder which is characterized by an increased gene expression in retinal ganglion cells.

It has not to date been possible in the prior art to provide such a molecule which makes it possible to treat such a disorder of the retina.

A further aspect of the present invention relates to a pharmaceutical composition which comprises the aforementioned nucleic acid molecule and a pharmaceutically acceptable carrier and, where appropriate, further excipients and active ingredients.

Pharmaceutically acceptable carriers and excipients are described in detail in the prior art; cf. for example Kibbe A. (2000), “Handbook of Pharmaceutical Excipients”, American Pharmaceutical Association and Pharmaceutical Press. The content of this publication is incorporated in the present application by reference. Such a composition may include further active ingredients which are advantageous in connection with the treatment of the disorder of the retina and are known to the skilled worker.

A further aspect of the present invention relates to a method for the targeted administration of a composition into the retinal ganglion cells (RGC) in a human or animal being, comprising the steps: (a) provision of a composition which includes nucleic acid molecules, and (b) injection of the composition into the superior colliculus of the being.

It will be appreciated that the features mentioned above and yet to be explained below can be used not only in the combination indicated in each case, but also in other combinations or alone, without departing from the scope of the present invention.

The present invention is now explained in more detail by means of exemplary embodiments which are purely illustrative and which do not in any way restrict the scope of the present invention. Reference is made in this connection to the appended figures, in which the following is to be seen:

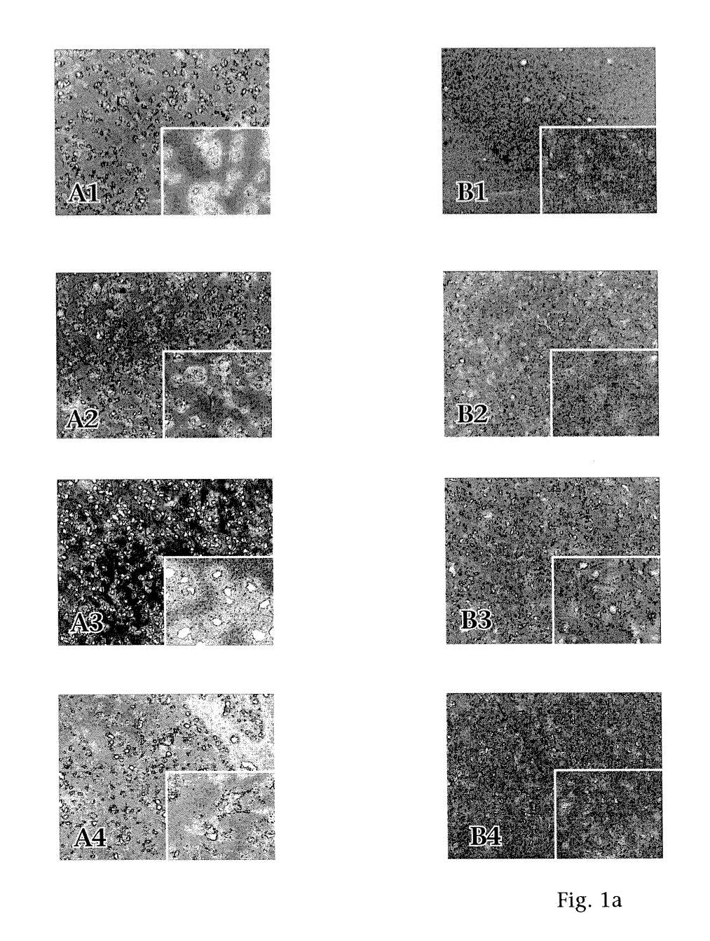

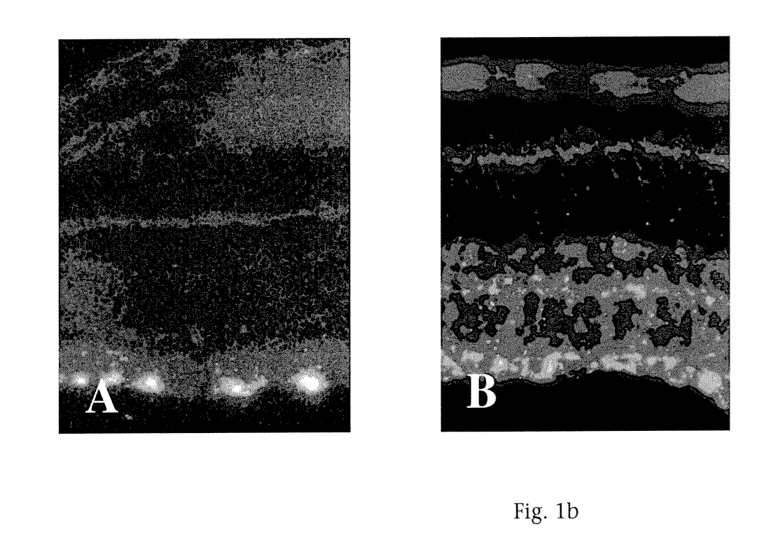

FIG. 1 shows by means of fluorescence microscopy investigations of transverse sections of the rat retina the targeted transport of nucleic acid molecules into retinal ganglion cells after injection into the superior colliculus and the nonspecific transport of nucleic acid molecules which have been injected into the vitreous body of a rat eye;

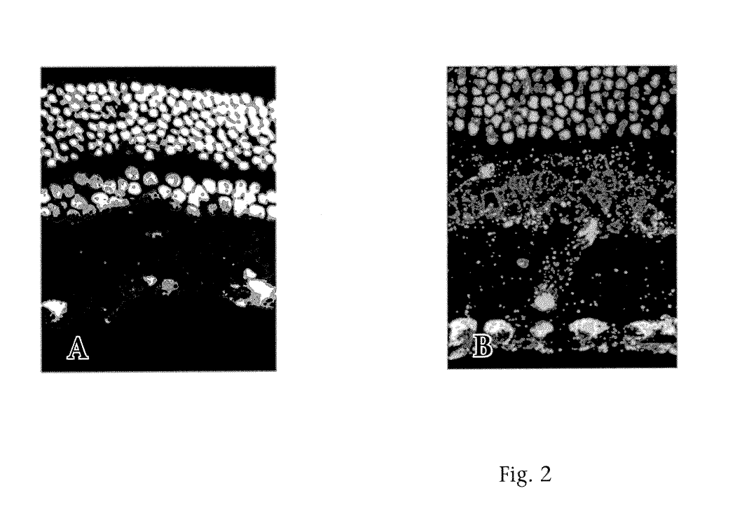

FIG. 2 shows by means of investigations of transverse sections of the rat retina using confocal laser scanning microscopy the specific transport of nucleic acid molecules into retinal ganglion cells which have been injected into the superior colliculus, whereas nucleic acid molecules injected intravitreally are transported into diverse retinal layers;

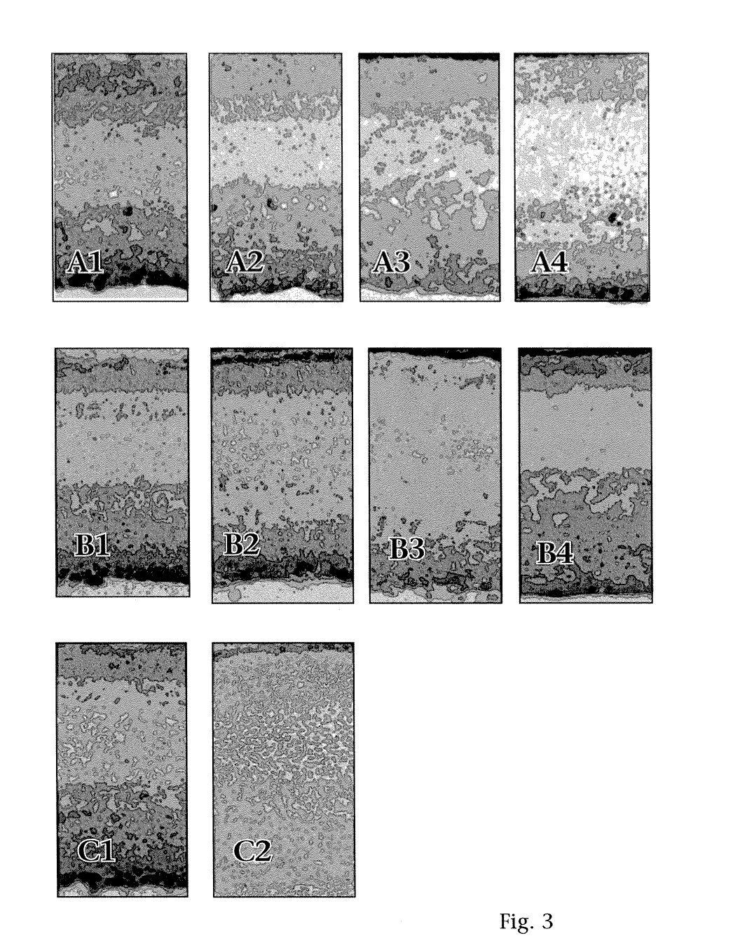

FIG. 3 shows by means of immunohistochemical investigations on transverse sections of the rat retina the inhibition of KAT II gene expression in retinal ganglion cells by a specific nucleic acid molecule which was injected into the superior colliculus;

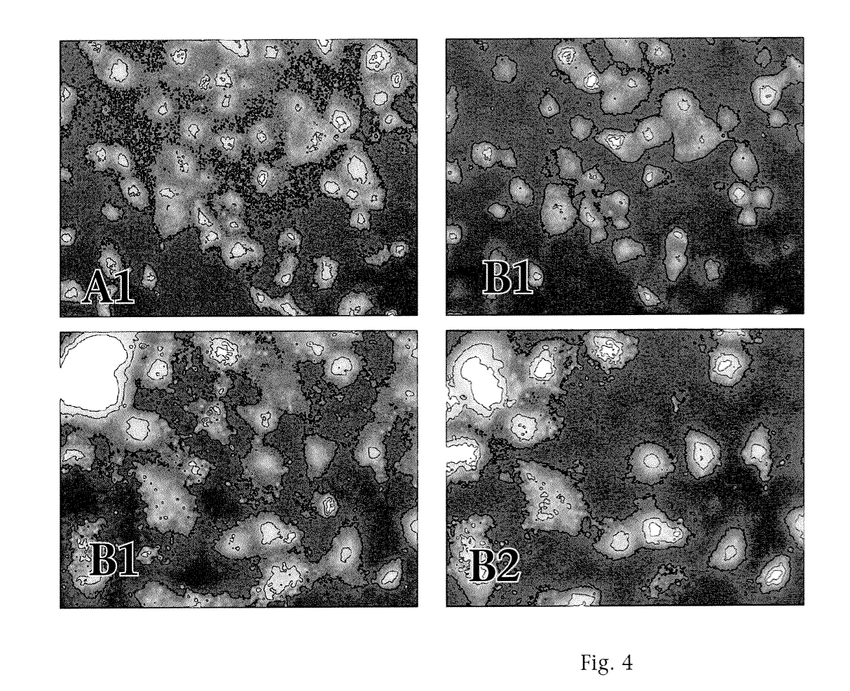

FIG. 4 shows by means of fluorescence microscopy investigations on flat-mounts of the rat retina a costaining of retinal ganglion cells both with fluorescence-labeled nucleic acid molecule and with Fluorogold-labeled nucleic acid molecule after injection thereof into the superior colliculus.

### EXEMPLARY EMBODIMENTS

### 1. Material and Methods

### Animals

All the experiments were carried out in compliance with the guidelines on the treatment of animals in the European Union and the ARVO (Association for Research in Vision and Opthalmology). The model animals used were brown Norway rats (Charles River, Wilmington, Mass., United States of America) with a body weight of from 150 to 200 g. The animals were kept with a 12-hour light-dark cycle and provided with feed and water ad libitum.

### Nucleic Acid Molecules

The nucleotide sequence for the KAT II mRNA was taken from the GenBank database; www.ncbi.nim.nih.gov/Genbank/. The sequence of the antisense oligonucleotide (ODN) against KAT II (KAT II ODN) was as follows: 5′-TTCATGTCTCTGCTGGTCGC-3′. This KAT II ODN configured as 20 bp oligonucleotide is complementary to the region of the start codon of the KAT II mRNA. An ODN with the following randomized sequence was used as control: 5′-GTACGTCTGTTCCTGTTCCG-3′. The ODNs were synthesized commercially, where appropriate as PS-ODN (PS: phosphorothiotate backbone) (biomers.net, Ulm, Germany).

Fluorescence-labeled or unlabeled PS-ODNs against KAT II, randomized ODN and NHSCy3-fluorescent dye alone as control were dissolved in ddH₂O (pH 7.4) with a final concentration of 100 μm for intravitreal injections and injections into the superior colliculus.

### Intravitreal Injections

Rats were anesthetized by an intraperitoneal injection of chloral hydrate (6 ml/kg of body weight in a 7% strength solution). The injection into the eyes took place using a heat-drawn glass capillary which was connected to a microinjector (Drummond Scientific Co., Broomall, Pa., United States of America) with direct microscopic observation. Animals with visible damage to the lenses were excluded from the experiments.

A single injection of 2 μl with 100 μM ODN or PS-ODN (equivalent to 200 pmol) was administered. The contralateral eyes served as control eyes into which the randomized oligonucleotides were injected.

### Tissue Preparation

For the immunohistochemistry, the animals were sacrificed with CO₂ 2 days, 6 days, 2 weeks and 6 weeks after the injections, and the eyes were immediately enucleated. After hemisection of the eyes along the ora serrata, the cornea, the lenses and the vitreous body were removed. The eye caps were fixed by immersion in 4% (weight/volume) paraformaldehyde in phosphate buffer (PB; 0.1 M pH 7.4) at 4° C. for 30 minutes. After washing in PB three times, the tissues were cryoprotected by immersion in 30% (weight/volume) sucrose in PB at 4° C. overnight. The samples were subsequently embedded in a cryomatrix (Jung, Leica, Heidelberg, Germany).

Radial sections 10 to 12 μm thick were cut out of the embedded eye caps using a cryostat, collected on silane-coated slides, air-dried and stored at 20° C. for further use.

Injection into the Superior Colliculus

Under deep anesthesia, 7 μl of the fluorescent marker hydroxystilbamidine methanesulfonate (Fluorogold, Molecular Probes, Eugene, Oreg., United States of America) were injected by three stereotaxic injections into the superior colliculus of the animals. 100 μm fluorescence-labeled or unlabeled ODNs or a combination of Fluorogold- and fluorescence-labeled ODNs were used. The animals were sacrificed with CO₂ 2 days, 6 days, 2 weeks and 6 weeks after the injection. The eyes were enucleated, the retinas were dissected out, flat-mounts on cellulose nitrate filters (pore size 60 μm; Sartorius, Long Island, N.Y., United States of America) were prepared and fixed in 2% paraformaldehyde for 30 minutes. Inspection took place immediately under a fluorescence microscope. Pictures were obtained, coded and analyzed through a digital imaging system which was connected to the microscope (ImagePro 3.0, Media Cybernetics Inc., Silver Spring, Md., United States of America).

### 2. Results

### 2.1 Injections of Cy3-Labeled PS-ODN

Following intravitreal injections of Cy3-labeled ODNs or injections into the superior colliculus, the transfection of the retina and of the retinal ganglion cells was observed after various times from one day up to two weeks using fluorescence microscopy on retinal flat-mounts and radial sections.

### Retrograde Transfection

Following retrograde injections of Cy3-labeled ODN into the superior colliculus of rats, RGC in retinal flat-mounts were analyzed 1 day (FIG. 1a, A1), 2 days (FIG. 1a, A2), 6 days (FIG. 1a, A3) or 2 weeks (FIG. 1a, A4) after the injection. This revealed an intense stain which remained intense over the whole period. In radial sections of the retina, even after up to 2 weeks no fluorescence was observed in other sections of the retina apart from the RGC layer (FIG. 1b, A, 2 days) (other times are not depicted).

As control, the fluorescent Cy3-NHS ester was injected alone (not coupled to ODN) into the superior colliculus. In this case, fluorescence was not detected anywhere in the whole retina (data not shown).

The nucleic acid molecules injected into the superior colliculus were accordingly transported highly selectively and exclusively into the RGC.

### Intravitreal Injections

Following intravitreal injections of Cy3-labeled PS-ODN in rats, fluorescence-labeled cells were detected in the RGC layer in the retinal flat-mounts after 1 day (FIG. 1a, B1), 2 days (FIG. 1a, B2), 6 days (FIG. 1a, B3) or 2 weeks (FIG. 1a, B4). Deeper layers of the retinal flat-mounts were likewise stained (FIG. 1a, B1 to B4, the pictures show the focus on the RGC layer). Investigation of radial sections revealed that cells of other layers of the retina, including the retinal pigment epithelium, were stained 2 days after injection (FIG. 1b, B).

This pattern remained unchanged over the whole period (other times are not shown).

The nucleic acid molecules injected into the vitreous body of the eyes were accordingly transported nonspecifically into a large number of tissues.

### 2. Laser Scanning Microscopy

It was possible by laser scanning microscopy to detect the fluorescent ODNs both in the cytoplasm and in the nucleus of the RGC 4 days after carrying out the injections by both methods. Following injections into the superior colliculus, exclusively RGC were transfected with ODNs (pale or red stain, FIG. 2, A). Following intravitreal injections, both RGC (pale or red) and other layers of the retina were transfected (blue stain, FIG. 2b) with ODNs.

The experiments described above are therefore confirmed.

### 2.3 Immunohistochemistry

Following injections of unlabeled specific KAT-II ODNs by both methods, the expression of KAT II in RGC was investigated at various times of up to one week (FIG. 3). In this case, downregulation of KAT II expression was to be observed only one day after the injection (FIG. 3, A2) and reached its maximum 3 days after injection into the superior colliculus (FIG. 3, A3). Following intravitreal injections, a comparable downregulation of KAT II expression was observed (FIG. 3, B2 to B4). The randomized ODN controls (FIG. 3, A1, B1) showed a stain which corresponds to that with untreated rats (FIG. 3, C1).

2.4 Costaining of RGC with Fluorescence-Labeled ODN and Fluorogold

Following injections (intravitreal and into the superior colliculus, associated with retrograde labeling of the RGC with Fluorogold FG), pictures were taken after monochromatic excitation of Cy3 (with length 450 nm) and FG (with length 360 nm) (FIG. 4).

Following injections of Cy3-ODN into the superior colliculus and labeling of RGC with FG, all retinal ganglion cells (defined as cells labeled with FG (FIG. 4 A2)) were stained with Cy3 in the same way (FIG. 4 A1). Apart from the FG-labeled RGC, no other cells showed any Cy3-positive stain. The pattern was comparable at various times from 3 days up to 2 months (data not shown). The detected Cy3 fluorescence was most intense 3 days after the injection, and the detected fluorescence signals were distinctly weaker 2 weeks after the injections. In contrast thereto, the FG fluorescence remained at approximately the same intensity over the entire period (data not shown).

Following intravitreal injections of Cy3-ODN and labeling with RGC with FG, all retinal ganglion cells (defined as cells labeled with FG (FIG. 4, B2)) were likewise labeled with Cy3 (FIG. 4, B1). In addition besides the RGC (FG-labeled), further cells were stained with Cy3 (FIG. 4, B1 compared with B2). This pattern remained unchanged at various times from 3 days up to 2 months (data not shown).

### 3. Conclusions

The inventors have been able to show that nucleic acid molecules designed for intravitreal injection into the mammalian eye are transported after their injection into all cell layers of the retina and accumulate therein, whereas nucleic acid molecules designed for injection into the superior colliculus are transported after their injection exclusively into the cell layer consisting of the retinal ganglion cells and accumulate therein. The inventors have been able to show further that solely the appropriate nucleic acid molecule or a composition containing the latter is specifically transported into the retinal ganglion cells after injection into the superior colliculus, but an uncoupled fluorescent dye injected in the same way is not.

The inventors accordingly provide a tool which is extremely valuable for therapy and diagnosis and with which active substances of a wide variety of types, but also diagnostic aids, which are coupled to the nucleic acid molecule can be administered into the RGC by a simple injection into the midbrain of a patient, whereas other cells of the retina or of the organism are substantially uninfluenced.

The inventors have been able to demonstrate further that gene expression in retinal ganglion cells can be modulated in a targeted manner by using specific nucleic acid molecules, for example antisense oligonucleotides or siRNA molecules, which can be injected into the midbrain. The nucleic acid molecule can therefore be configured in such a way that it has therapeutic activity itself.

1. A method for targeted administration of a composition into retinal ganglion cells (RGC) of a human or animal being comprising the steps of:
(a) providing the composition to be administered into the RGC; and
(b) injecting the composition into the superior colliculus of the human or animal being, wherein the composition comprises a nucleic acid molecule.

2. (canceled)

3. The method of claim 1, wherein an active ingredient is coupled to the nucleic acid molecule.

4. The method of claim 1, wherein the nucleic acid molecule is designed as an active ingredient.

5. The method of claim 1, wherein the nucleic acid molecule modulates gene expression in the RGC.

6. The method of claim 1, wherein the nucleic acid molecule has a nucleotide sequence which is complementary to the sequence of an mRNA from the RGC.

7. The method of claim 6, wherein the mRNA encodes a gene or protein which is selected from the group consisting of: c-fos, c-jun, p53, Bax, Apafl, caspase 9, caspase 3, caspase 6, and PARP.

8. The method of claim 1, wherein the nucleic acid molecule is a siRNA.

9. The method of claim 1, wherein the nucleic acid molecule is designed so that it binds to another nucleic acid molecule which encodes a kynurenine aminotransferase II (KAT II) or parts thereof.

10. The method of claim 1, wherein the nucleic acid molecule comprises the nucleotide sequence TTCATGTCTCTGCTGGTCGC (SEQ ID NO. 1).

11. The method of claim 1, wherein a detectable marker is coupled to the nucleic acid molecule.

12.-14. (canceled)

15. The method of claim 5, wherein the nucleic acid molecule modulates gene expression in the RGC by inhibiting gene expression in the RGC.

16. A method for treating an ocular disorder which is characterized by increased gene expression in retinal ganglion cells (RGC), comprising:
(a) providing an effective amount of composition to be administered into the RGC; and
(b) injecting the composition into the superior colliculus of the human or animal being, wherein the composition comprises a nucleic acid molecule, thereby treating the ocular disorder which is characterized by increased gene expression in retinal ganglion cells (RGC).

17. The method of claim 16, wherein an active ingredient is coupled to the nucleic acid molecule.

18. The method of claim 16, wherein the nucleic acid molecule is designed as an active ingredient.

19. The method of claim 16, wherein the nucleic acid molecule modulates gene expression in the RGC.

20. The method of claim 19, wherein the nucleic acid molecule modulates gene expression in the RGC by inhibiting gene expression in the RGC.

21. The method of claim 16, wherein the nucleic acid molecule has a nucleotide sequence which is substantially complementary to the sequence of an mRNA from the RGC.

22. The method of claim 21, wherein the mRNA encodes a gene or protein which is selected from the group consisting of: c-fos, c-jun, p53, Bax, Apafl, caspase 9, caspase 3, caspase 6, and PARP.

23. The method of claim 16, wherein the nucleic acid molecule is a siRNA.

24. The method of claim 16, wherein said nucleic the molecule is designed so that it binds to another nucleic acid molecule which encodes a kynurenine aminotransferase II (KAT II) or parts thereof.

25. The method of claim 16, wherein the nucleic acid molecule comprises the nucleotide sequence TTCATGTCTCTGCTGGTCGC (SEQ ID NO: 1).

26. The method of claim 16, further comprising formulating the nucleic acid molecule into a pharmaceutically acceptable carrier.

27. The method of claim 16, wherein a detectable marker is coupled to the nucleic acid molecule.

28. An injectable pharmaceutical composition which is transported into retinal ganglion cells (RGC) in a targeted fashion, comprising a nucleic acid molecule and a pharmaceutically acceptable carrier.

29. The injectable pharmaceutical composition of claim 28 wherein an active ingredient is coupled to the nucleic acid molecule.

30. The injectable pharmaceutical composition of claim 28 wherein the nucleic acid molecule is designed as an active ingredient.

31. The injectable pharmaceutical composition of claim 28 wherein the nucleic acid molecule modulates gene expression in the RGC.

32. The injectable pharmaceutical composition of claim 28, wherein the nucleic acid molecule modulates gene expression in the RGC by inhibiting gene expression in the RGC.

32. The injectable pharmaceutical composition of claim 28 wherein the nucleic acid molecule has a nucleotide sequence which is substantially complementary to the sequence of an mRNA from RGC.

34. The injectable pharmaceutical composition of claim 33 wherein the mRNA encodes a gene or protein which is selected from the group consisting of: c-fos, c-jun, p53, Bax, Apafl, caspase 9, caspase 3, caspase 6, and PARP.

35. The injectable pharmaceutical composition of claim 28 wherein the nucleic acid molecule is a siRNA.

36. The injectable pharmaceutical composition of claim 28 wherein the nucleic acid molecule is designed so that it binds to another nucleic acid molecule which encodes a kynurenine aminotransferase II (KAT II) or parts thereof.

37. The injectable pharmaceutical composition of claim 28 wherein the nucleic acid molecule comprises the nucleotide sequence TTCATGTCTCTGCTGGTCGC (SEQ ID NO:1).

38. The injectable pharmaceutical composition of claim 28 wherein a detectable marker is coupled to the nucleic acid molecule.
